(19) United States
(12) Patent Application Publication
(10) Pub. No.: US 2021/0187257 A1
(43) Pub. Date: Jun. 24, 2021

(54) CRANIOSPINAL SURGICAL DRAIN

(71) Applicant: NeuroVention LLC, Daytona Beach, FL (US)

(72) Inventor: Rohit Khanna, Daytona Beach, FL (US)

(21) Appl. No.: 17/126,795

(22) Filed: Dec. 18, 2020

Related U.S. Application Data
(60) Provisional application No. 62/950,503, filed on Dec. 19, 2019.

(51) Int. Cl. A61M 27/00; A61M 1/00; A61M 25/00

(52) U.S. Cl. CPC A61M 27/00; A61M 1/0072; A61M 2210/1003; A61M 25/0068; A61M 2210/0693; A61M 1/0011

(57) ABSTRACT

A central nervous system drain drains the subdural space intracranially or the postoperative epidural space in the spine. The drain includes one or more lumens that communicate with the outside environment through openings in the distal portion of the drain. The drain also includes a flat bottom wall without any openings which is placed on the brain surface in the subdural space intracranially or the epidural surface in the spinal canal after a laminectomy. The openings in the top wall and/or side surface walls allow for drainage of fluid and/or blood in the subdural space or postoperative wound in the spine.

### BACKGROUND

The central nervous system comprises of the brain and spinal cord surrounded by the cerebrospinal fluid, arachnoid, dura and skull bone or spinal canal bone. A subdural hemorrhage comprises of blood accumulation in the space between the dura and the brain. A subdural hemorrhage causes compression of the underlying brain and increases intracranial pressure with associated neurologic deficits and, in severe cases, can lead to coma and death.

Treatment of a subdural hemorrhage involves the creation of a small hole in the skull termed a burr hole or a larger opening with a replaceable bone flap termed a craniotomy followed by drainage of the subdural hemorrhage. Not infrequently, the compressed brain from the subdural hemorrhage does not expand immediately creating an empty subdural space with an increased risk for recurrent hemorrhage or effusion (subdural hygroma). Subdural drains are frequently used to remove recurrent blood or fluid collection and allow for brain expansion. These drains facilitate drainage into an external collection bag either passively through gravity or a vacuum suction effect. Subdural drainage with suction has shown to be more effective than passive gravity drainage in facilitating brain re-expansion and preventing recurrent collections. A variety of drainage tubes and catheters have been devised for removing hemorrhage and excess fluids from postsurgical sites or wounds. Such drains often employ external grooves to collect the fluids, or contain perforations through which fluids can enter a central passageway that carries the fluids away from the site. One problem with existing drainage tubes and catheters is that with placement of the drain between the brain surface and the dura (subdural space), the brain surface tissue and/or blood vessels can often be suctioned into the drain, especially into its perforations, thus leading to brain damage with cortical contusions and recurrent bleeding, and also hindering drain removal. Thus, an optimal drain design is needed that efficiently removes subdural hemorrhage and/or fluids without the potential for suctioning brain tissue or surface vessels into the drain. The prior art drain designs have not addressed this problem.

After a laminectomy in the spine, the dura is closed in a watertight fashion with sutures. Frequently, drains are placed in the epidural space to remove any post-operative epidural hemorrhage accumulation. All prior art drains with circumferential holes allow suction over the sutured dura through negative suction pressure, which is used to remove the epidural hemorrhage. These drains increase the risk for cerebrospinal fistula formation and can create a cerebrospinal fluid leak. What is needed, therefore, is a drain that prevents this complication by avoiding a direct negative suction on the sutured dura surface.

In light of the above, there remains a need for a new and improved drain design for brain and spine post-operative drainage, particularly drains that minimize or eliminate the problem of occlusion of the drain by debris in a wound or the ingrowth of living tissue surrounding the drain, especially the brain and/or brain surface vessels.

### SUMMARY

The drain of the present disclosure is designed for central nervous system treatment and, in particular, for drainage of the subdural space intracranially and the postoperative epidural space in the spine. The drain comprises one or more lumens that communicate with the outside environment through perforations (i.e., holes or openings) in the distal portion of the drain wall which is placed in the central nervous system. The proximal portion of the drain is external and connected to a vacuum bulb for drainage of intracranial or intraspinal fluid or blood through a suction effect.

The drain also comprises a flat bottom wall without any perforations which is placed on the brain surface in the subdural space intracranially or the epidural surface in the spinal canal after a laminectomy. The flat bottom wall of the drain when placed in the subdural space over the brain surface does not comprise of any perforations (i.e., holes or openings) and, therefore, prevents any suction effect on the underlying brain or blood vessels, thereby preventing any injury and drain occlusion. The flat bottom wall when placed overlying the dura/durotomy in the spine also prevents any direct suction effect and leakage of any cerebrospinal fluid through the durotomy. The drain wall perforations in the top surface and/or side surface/walls allows for drainage of fluid and/or blood in the subdural space or postoperative wound in the spine. The drain internal lumen can also comprise of triangular teeth shaped ridges on the bottom surface which prevent the top wall from collapsing and occluding the drain lumen due to the suction effect. In another embodiment of the drain, an internal lumen wall also prevents the lumens from collapsing from the intraluminal negative pressure vacuum suction effect.

### BRIEF DESCRIPTION OF THE DRAWINGS

FIG. 1 is a schematic view of a drain in the subdural space over the brain and under the dura.

FIG. 2 is a schematic view of a first embodiment of the drain.

FIG. 3 is a partial sectional view of the first embodiment of the drain.

FIG. 4 is a cross-sectional view of the first embodiment of the drain along line IV-IV in FIG. 3.

FIG. 5 is a partial sectional view of a modified version of the first embodiment of the drain.

FIG. 6 is a cross-sectional view of the modified version of the first embodiment of the drain along line VI-VI in FIG. 5.

FIG. 7 is a schematic view of a second embodiment of the drain.

FIG. 8 is a cross-sectional view of the second embodiment of the drain.

FIG. 9 is a schematic view of the first embodiment of the drain in the subdural space over the brain and under the dura.

FIG. 10 is a schematic view of a third embodiment of the drain in the epidural space over the dura in the spine.

FIG. 11 is a cross-sectional view of the third embodiment of the drain in the epidural space over the dura in the spine.

FIG. 12 is a schematic view of a fourth embodiment of the drain.

FIG. 13 is a cross-sectional view of the fourth embodiment of the drain.

FIG. 14 is a schematic view of a fifth embodiment of the drain.

FIG. 15 is a cross-sectional view of the fifth embodiment of the drain.

### DETAILED DESCRIPTION

As shown in FIG. 1, a proximal portion 80 of a first embodiment of a drain 1, which is flexible, is placed inside the skull 2 in the subdural intracranial space 5 between the brain 3 and the dura 4. A distal portion 6 of the drain 1 is external and connected to a suction bulb 7. The suction bulb 7 has an in port 9 connected to the distal portion 6 of the drain 1 acting as a coaxial tube and an out port 8 with a cap that is used to empty fluid and/or blood accumulated in the suction bulb 7. The suction bulb 7 when compressed manually, creates a negative suction pressure through the in port 9 to allow the suction of fluid and/or blood from the drain 1. The in port 9 also has a one way valve that only allows fluid from the drain 1 to flow inside the suction bulb 7 and not allow any backflow into the subdural space. The suction bulb 7 can be compressed to create a negative pressure suction or left inflated as a drainage bag for gravity drainage without a negative suction pressure.

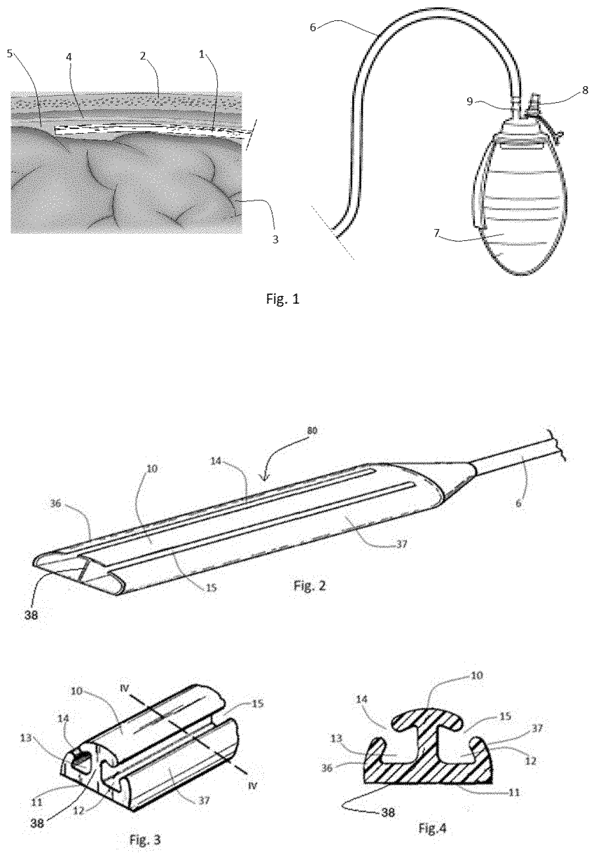

As shown in FIGS. 2-4, the proximal portion 80 of the drain 1, which is an elongated flexible tube having an internal passageway, comprises a flat bottom wall 11 and a convex top wall 10 with lumens 12 and 13. The side walls 36 and 37 extend from the top wall 10 to the bottom wall 11. The side walls 36 and 37, which can also have a convex (i.e., curved) shape, comprise longitudinal openings 14 and 15 (i.e., open channels) that provide communication of the outside environment with the lumens 13 and 12, which are separated by an internal wall 38. The flat bottom wall 11 is solid and does not comprise of any openings. The flat bottom wall 11 when placed on the surface of the brain or spinal cord in the subdural space or over the sutured dura in the epidural space does not allow a direct negative suction pressure and avoids suction of the brain or dura surface, thereby limiting brain injury and cerebrospinal fluid leak complications. The distal portion 6 of the drain 1 resides outside the central nervous system and is connected to the negative pressure suction bulb 7. The internal wall 38 prevents the lumens 12 and 13 from collapsing with the negative suction effect, thereby preventing drain obstruction.

It is also noted that instead of the longitudinal openings 14 and 15 being provided in the side walls 36 and 37, the longitudinal openings 14 and 15 can be provided in the top wall 10. Further, each of the longitudinal openings 14 and 15 can be replaced with a row of openings, which can be round, extending in the longitudinal direction.

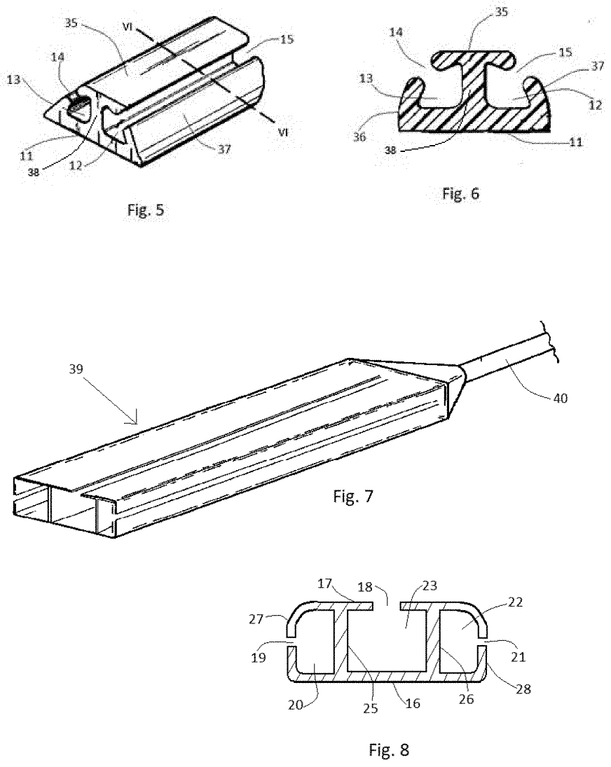

The top wall 10 can be convex to contour to the convex skull anatomy as shown in FIGS. 3 and 4. In a modified version of the first embodiment, the top wall 10 can be changed to a top wall 35 that is flat as shown in FIGS. 5 and 6. The top wall 10, 35 can also be solid without any holes or openings.

The drain 1 when placed in the subdural space has the bottom wall 11 on the surface of the brain 3 and the top wall 10, 35 on the dura 4 and overlying skull 2. Since the top and bottom walls do not comprise of any holes (i.e., openings) the desired effect of no direct suction on the brain surface or the dura is achieved. The longitudinal openings 14 and 15 in the side walls 36 and 37 allow fluid and/or blood drainage from the subdural space between the brain 3 and the dura 4. The larger outer dimension (i.e., width) of the bottom wall 11 relative to the top wall 10, 35 and the side walls 36 and 37 prevents the brain 3 from being injured by being suctioned into the longitudinal openings 14 and 15 with the suction vacuum effect. The wider bottom wall 11 also prevents the drain 1 from turning on its sides or rotating and thereby prevents a direct suction effect on the underlying brain 3.

Another protective feature of the drain design is from the longitudinal openings 14 and 15 being in the side walls 36 and 37 that prevent the brain 3 or brain surface vessels under the bottom wall 11 from being suctioned into the longitudinal openings 14 and 15. thereby avoiding any brain or blood vessel injury.

In a second embodiment as shown in FIGS. 7 and 8, the drain 1 comprises a proximal portion 39 placed inside the central nervous system and a distal portion 40 connected to the suction bulb 7. The proximal portion 39 of the drain 1 comprises a bottom wall 16, side walls 27 and 28, and a top wall 17. The top wall 17 comprises an opening 18 that provides communication between the outside environment and a middle lumen 23. The side walls 27 and 28, which have a flat shape, also comprise openings 19 and 21 that provide communication of the outside environment with lumens 20 and 22, respectively, which are separated from the middle lumen 23 by internal walls 25 and 26. The bottom wall 16 is solid and does comprise of any openings. The internal walls 25 and 26 prevent collapsing of the lumens 20, 22 and 23 with a negative suction effect, thereby preventing drain obstruction.

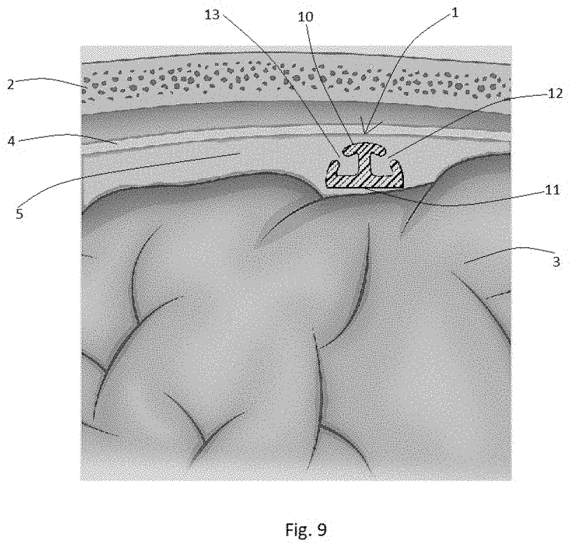

FIG. 9 illustrates the proximal portion 80 of the first embodiment of the drain 1 placed in the intracranial subdural space 5 between the dura 4 and the brain 3. The skull 2 has a convex anatomy that correlates with the convex top wall 10 of the drain 1. The flat bottom wall 11 of the drain 1 has no holes and when placed on the surface of the brain 3 avoids any suction on the brain 3 or the cortical surface blood vessels. The longitudinal openings 14 and 15 in the convex side walls 36 and 37 communicate with the subdural space and provide for drainage of any excess fluid or blood into the drain 1 with negative suction pressure transmitted through the lumens 12 and 13. The larger flat bottom wall 11 along with the side walls 36 and 37 reduce the risk of direct suction injury of the underlying brain 3 and blood vessels.

The drain 1 can also be placed in the spinal epidural space. After a laminectomy in the spine with intradural exposure, the dura is closed in a watertight fashion with sutures. Since the drain surface in contact with the closed dura does not have any holes for a direct negative suction on the dura surface, this avoids the risk of creating a cerebrospinal fluid leak and/or a cerebrospinal fistula formation.

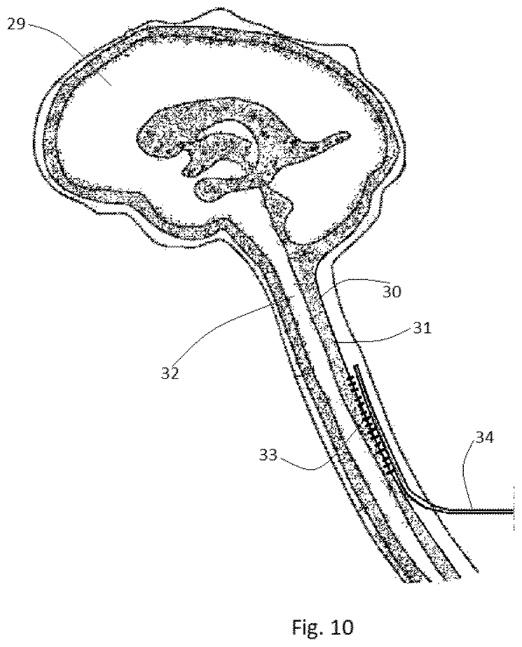

FIG. 10 illustrates a third embodiment of the drain (i.e., a drain 34) placed in the spine in the epidural space. The intrathecal space 30 contains the cerebrospinal fluid which surrounds the brain 29 and the spinal cord 32 with the outer portion comprising of the dura 31. Following an intradural spine procedure, the dura 31 is closed with sutures 33. The drain 34 is placed in the epidural space to remove any post-operative hemorrhage and/or fluid.

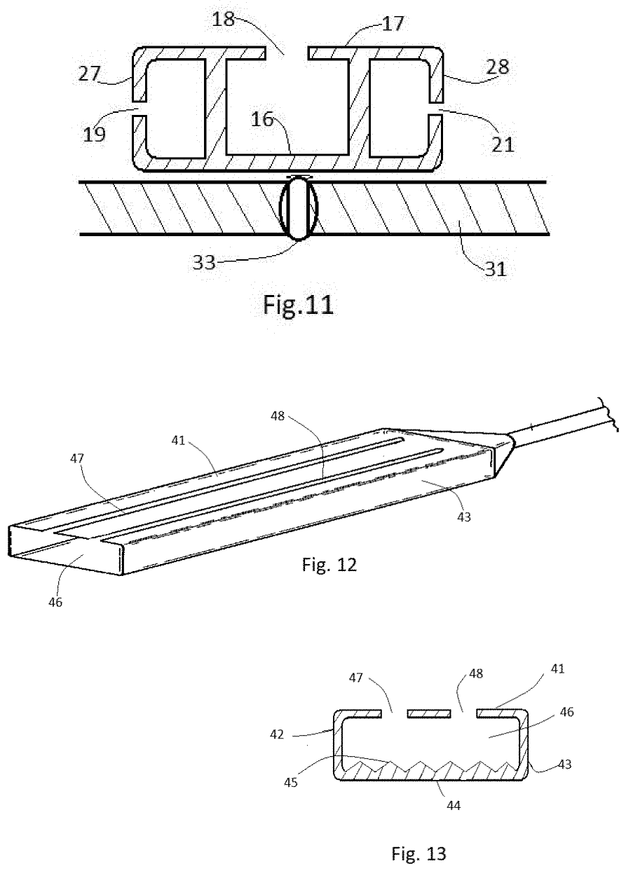

As shown in FIG. 11, the drain 34 is placed in the epidural space over the dura 31 and dura sutures 33. The drain bottom surface 16 has no openings and when placed on the surface of the dura 31, it avoids any direct suction on the dura 31 and the creation of a cerebrospinal fluid leak or fistula formation. The top wall 17 and the side walls 27 and 28 have openings 18, 19 and 21 that suction the excess fluid and/or blood from the epidural space.

In a fourth embodiment as shown in FIGS. 12 and 13, the drain comprises a top wall 41, side walls 42 and 43, and bottom wall 44 which all have a flat surface. The top wall 41 comprises two longitudinal openings 47 and 48 that communicate the outside environment with the lumen 46. The bottom wall 44 also comprises of ridges 45 extending into the lumen 46. The ridges 45 prevent the walls from collapsing and completely occluding the lumen 46 with negative suction effect thereby preventing the drain from obstruction.

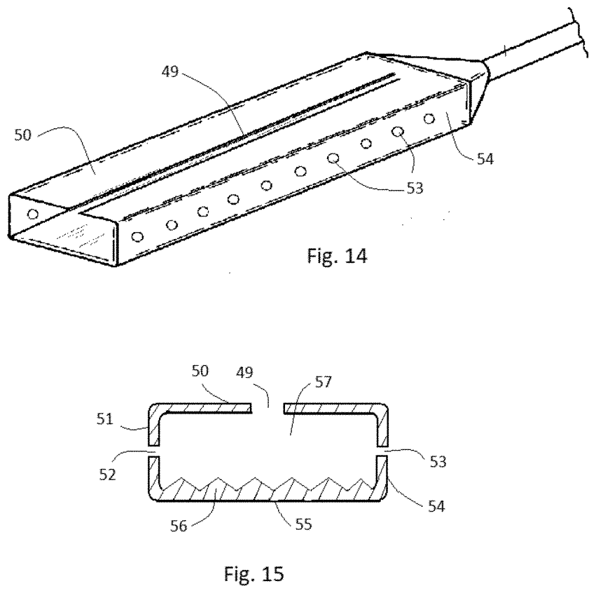

In a fifth embodiment as shown in FIGS. 14 and 15, the drain comprises a top wall 50, side walls 51 and 54, and bottom wall 55 which all have a flat surface. The top wall 50 comprises of a longitudinal opening 49 that communicates the outside environment with a lumen 57. The side walls 51 and 54 comprise multiple round openings 52 and 53 (i.e., circular holes) that provide communication from the outside environment into the lumen 57. The small side wall openings 52 and 53 prevent the underlying brain or blood vessels from being suctioned into the lumen 57 with a negative suction effect. The bottom wall 55 also comprises ridges 56 extending into the lumen 57. The ridges 56 prevent the walls from collapsing and completely occluding the lumen 57 due to a negative suction effect, thereby preventing the drain from obstruction.

The drain described provides for treatment of any central nervous system pathology including, but not limited to, treatment of increased intracranial pressure, brain swelling or edema, spinal cord edema, trauma, brain injury, skull fracture, stroke, ischemia, hypoxia following respiratory or cardiac arrest, tumors, hemorrhage, infection, seizure, spinal cord injury, spine fractures, arteriovenous malformations, aneurysms, aortic artery surgery related spinal cord ischemia protection, thoracic and/or abdominal aortic aneurysm or dissection surgical or endovascular repair, spinal stenosis, herniated disc, and scoliosis surgery.

The drain can be placed intracranially following the drilling of a hole in the skull via a twist drill, burr hole placement, or craniotomy/craniectomy. It can be placed inside the spinal canal in the epidural, subdural or subarachnoid space through a percutaneous technique or following a laminotomy/laminectomy. The placement of the drain intracranially or intraspinally can be further facilitated by radiographic guidance (fluoroscopy), ventriculograms, cisternograms, myelogram with injection of contrast agent through the catheter, ultrasound, frame based or frameless stereotactic navigation systems, or endoscopy. The drain can also comprise radio-opaque markers or be impregnated with barium to visualize correct placement in the central nervous system with x-rays. Other locations include in the surgical resection bed following a craniotomy for removal of a brain tumor or hemorrhage and the spinal epidural or intrathecal space following a laminectomy. The drain can also be placed extracranially under the scalp overlying the burr hole in the subgaleal or subperiosteal space for drainage of the subdural space through the burr hole.

The flexible drain may be fabricated from materials known in the art including, but not limited to, aliphatic polyamides, fluorinated ethylene propylene, nylon, perfluoroalkoxy (e.g., Teflon®), polyether block amide)(Pebax®, polyetheretherketone (PEEK), polyethylene (e.g.) Tyvek®, polytetrafluoroethylene (PTFE), polypropylene, polyurethane, polyvinylchloride, natural rubber, nitrile rubber, silicone rubber, combinations and copolymers thereof, and the like. The flexible drain walls may be transparent, translucent, or opaque and the surfaces may be smooth, textured, or a combination thereof. Furthermore, the drain wall may be impermeable or semipermeable to materials including, but not limited to, gases, liquids, proteins or molecules of a given size or range of sizes, combinations thereof, and the like.

While the embodiments of the drain described herein along with the illustrations are specific, it is understood that the drain is not limited to the embodiments disclosed. Numerous modifications, rearrangements, and substitutions can be made with those skilled in the art without departing from the spirit of the disclosure as set forth and defined herein. For example, any feature of any of the embodiments of the drain can be combined with any other feature(s) of any of the other embodiments of the drain. It is also understood that the drain is not limited to the central nervous system use and can also be used for other locations in the human body like the heart pericardial area to avoid direct suction on the coronary vessels, the chest cavity pleural space to avoid direct suction on the lung surface, the neck after a carotid endarterectomy to avoid direct suction on the carotid artery suture line, vessels in the body after vascular surgery, etc.

What is claimed is:

1. A drain for removing fluids from within a body, the drain comprising:
an elongate flexible tube having an internal passageway surrounded by a top wall, a bottom wall, and two side walls; and
one or more walls that divide the internal passageway into two or more lumens,
wherein the side walls comprises one or more openings communicating from outside the drain into the lumens, and
wherein the bottom wall is solid without any openings.

2. The drain of claim 1, wherein the one or more openings comprises round openings.

3. The drain of claim 1, wherein the one or more openings comprise open channels along a longitudinal length of the elongated flexible tube.

4. The drain of claim 1, wherein the top wall comprises a flat shape.

5. The drain of claim 1, wherein the bottom wall comprises a flat shape.

6. The drain of claim 1, wherein the side walls comprise a flat shape.

7. The drain of claim 1, wherein the top wall comprises a convex shape.

8. The drain of claim 1, wherein the side walls comprise a curved shape.

9. The drain of claim 1, wherein the top wall and the side walls each comprise an outer dimension that is smaller than an outer dimension of the bottom wall.

10. The drain of claim 1, wherein the drain is connected to a drainage bag with a coaxial tube.

11. The drain of claim 10, wherein the drainage bag comprises a vacuum suction bulb.

12. The drain of claim 1, wherein the top wall also comprises at least one opening.

13. The drain of claim 1, wherein the top wall is solid without any openings.

14. A drain for removing fluids from within a body, the drain comprising:
an elongate flexible tube having an internal passageway with a flat top wall, a flat bottom wall, and convex side walls,
wherein the convex side walls comprise openings communicating from outside the drain into the internal passageway,
wherein the internal passageway is divided into two lumens by an internal wall between the openings, and
wherein the flat top wall and the flat bottom wall are solid without any openings.

15. The drain of claim 14, wherein the openings comprise round openings

16. The drain of claim 14, wherein the openings comprise open channels along a longitudinal length of the elongated flexible tube.

17. The drain of claim 14, wherein an outer dimension of the flat top wall is smaller than an outer dimension of the flat bottom wall.

18. The drain of claim 14, wherein the drain is connected to a drainage bag with a coaxial tube.

19. The drain of claim 18, wherein the drainage bag comprises a vacuum suction bulb.

20. A drain for removing fluids from within a body, the drain comprising:
an elongate flexible tube having an internal passageway with a flat bottom surface and a convex top surface,
wherein the convex top surface comprises two rows of openings communicating from outside the drain into the internal passageway,
wherein the internal passageway is divided into two lumens by an internal wall between the two rows of openings, and
wherein the flat bottom surface is solid without any opening.

21. The drain of claim 20, wherein the openings comprise round openings.

22. The drain of claim 20, wherein the openings comprise open channels along a longitudinal length of the elongated flexible tube.

23. The drain of claim 20, wherein the drain is connected to a drainage bag with a coaxial tube.

24. The drain of claim 23, wherein the drainage bag comprises a vacuum suction bulb.
